(19) United States
(12) Patent Application Publication
(10) Pub. No.: US 2015/0065829 A1
(43) Pub. Date: Mar. 5, 2015

(54) SYSTEMS AND METHODS FOR RESPIRATION MONITORING

(71) Applicant: Nellcor Puritan Bennett Ireland, Mervue (IE)

(72) Inventors: Paul Stanley Addison, Edinburgh (GB); James N. Watson, Dunfermline (GB); Scott McGonigle, Edinburgh (GB)

(21) Appl. No.: 14/535,835

(22) Filed: Nov. 7, 2014

Related U.S. Application Data
(63) Continuation of application No. 12/508,172, filed on Jul. 23, 2009.

(51) Int. Cl. A61B 5/0205; A61B 5/00; A61B 5/113; A61B 5/1455

(52) U.S. Cl. CPC A61B 5/0205; A61B 5/14551; A61B 5/684; A61B 5/7278; A61B 5/6823; A61B 5/6822; A61B 5/726; A61B 5/113; A61B 5/085
USPC 600/324

(57) ABSTRACT

According to embodiments, techniques for determining respiratory parameters are disclosed. More suitable probe locations for determining respiratory parameters, such as respiration rate and respiratory effort, may be identified. The most suitable probe location may be selected for probe placement. A scalogram may be generated from the detected signal at the more suitable location, resulting in an enhanced breathing band for determining respiratory parameters. Flexible probes that allow for a patient's natural movement due to respiration may also be used to enhance the breathing components of the detected signal. From the enhanced signal, more accurate and reliable respiratory parameters may be determined.

### CROSS REFERENCE TO RELATED APPLICATION

This application is a continuation of U.S. patent application Ser. No. 12/508,172 filed Jul. 23, 2009, which is hereby incorporated by reference herein in its entirety.

### SUMMARY

The present disclosure relates to signal processing and, more particularly, the present disclosure relates to processing, for example, a photoplethysmograph (PPG) signal to determine respiratory parameters or other physiological parameters of a patient.

In an embodiment, at least one probe (e.g., a pulse oximeter probe) is positioned on a patient's body at a location suitable for determining a respiratory parameter, such as respiration rate or respiratory effort. For example, as described in more detail in U.S. Patent App. Pub. No. 2006/0258921, which is incorporated by reference herein in its entirety, the act of breathing may cause a breathing band to become present in a scalogram derived from a continuous wavelet transform of a PPG signal. This breathing band may occur at or about the scale having a characteristic frequency that corresponds to the breathing frequency. Furthermore, the features within this band (e.g., the energy, amplitude, phase, or modulation) or the features within other bands of the scalogram may result from changes in breathing rate (or breathing effort) and therefore may be correlated with various respiratory parameters of a patient.

A suitable location for positioning the at least one probe may include, for example, one or more locations where at least one breathing component of the signal detected by the at least one probe is stronger than at least one non-breathing component of the detected signal (e.g., one or more pulse components). In wavelet space, the more suitable locations for detecting respiratory parameters may include locations where the energy associated with the breathing band exceeds the energy associated with the pulse band (or the ratio of breathing band energy to pulse band energy exceeds a threshold ratio). Because a strong pulse band (and hence high pulse band energy) may distort or interfere with the breathing components in the detected signal, in an embodiment, the at least one probe may also be positioned at a location where the detected pulse band energy is less than a threshold energy level.

In an embodiment, probe locations may be selected where the modulation of the venous component dominates the PPG signal (or exceeds the arterial pulsatile component). Additionally or alternatively, locations exhibiting movement or motion associated with respiration may also be selected as more suitable probe locations to determine a patient's respiratory parameters. These locations may include, for example, a patient's collarbone, abdomen, side, chest (e.g., on or near the upper pectoral muscle), back, shoulder, or neck.

In an embodiment, an ideal probe location may be determined by testing multiple candidate locations on a patient's body (e.g., one or more of the patient's collarbone, abdomen, side, chest, back, shoulder, and neck). At each tested location, an index may be generated and outputted to a user (e.g., a physician or technician). The index may be outputted in visual or audible form and may be proportional to, for example, the breathing band energy, the ratio of the breathing band energy to the pulse band energy, or the pulse band energy. In an embodiment, the location associated with the greatest index may be selected as the ideal probe location for determining respiratory parameters.

In an embodiment, the at least one probe may include at least one wireless pulse oximetry probe in wireless communication with a parent system (e.g., pulse oximetry system or other physiological characteristic monitoring system). The at least one wireless probe may be attached (e.g., using removable adhesive, gel, or a suction cup attachment) to a patient at a suitable location for determining respiratory parameters. In this way, no extra lead may be required to monitor respiratory parameters.

Multiple wireless probes may also be used in some embodiments. One or more of the wireless probes may be pulse oximeter probes. One wireless probe may be positioned at a more traditional location for pulse oximetry (e.g., on a finger or toe) and used to determine a patient's blood oxygen saturation (referred to as a “SpO₂” measurement), while another wireless probe may be placed at a more suitable location for determining respiratory parameters. Multiple additional wireless probes may also be positioned at other locations to determine various other physiological parameters. For example, one wireless probe may be positioned on the finger and used to determine SpO₂, one wireless probe may be positioned on the abdomen and used to determine respiration rate, one wireless probe may be positioned on the chest and used to determine respiratory effort, and one wireless probe may be positioned on the ear (or finger) and used to determine blood pressure. Non-invasive systems and methods for determining blood pressure are described in more detail in U.S. patent application Ser. No. 12/242,238, which is incorporated by reference herein in its entirety.

In an embodiment, a probe configuration (referred to herein as a “flexible probe”) for use in determining respiratory parameters (e.g., respiration rate and respiratory effort) is provided. This probe configuration may allow for the natural movement due to respiration at certain locations on a patient's body to enhance the respiratory component in a detected PPG signal (or the respiration band in the scalogram derived from the PPG signal). The probe may include at least one energy emitting source (e.g., a light emitting source) separated from an energy detector or sensor (e.g., a photodetector) by a flexible member. The flexible member may allow the housing for the energy emitting source to move relative to the housing for the energy detector or sensor. Surfaces of a patient's body that move in phase with the patient's breathing may then enhance the respiratory component of the detected signal (e.g., the PPG signal). One or more additional energy emitting sources may be rigidly attached (or included within) the housing of the energy detector or sensor.

In an embodiment, the flexible probe configuration may include multiple energy detectors or sensors in a flexible array that covers a local area within the vicinity of one or more energy emitting sources. In this way, a plurality of signals may be detected and indicative of motion within a local area on the patient's body. A single probe may also be used to detect both SpO₂ and respiratory parameters by positioning at least one of the energy emitting sources on the same rigid substrate as at least one energy emitting source.

In an embodiment, the flexible probe configuration may include a flexible member that is restrained from moving in one or more planes of motion. For example, a pivot may be used to restrain horizontal motion (e.g., between the energy emitting source and energy detector or sensor) and allow for vertical motion (or restrain vertical motion and allow for horizontal motion). The planes of permitted and restrained motion may be used increase the resolution or energy associated with the respiratory components of the detected signal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these newly derived signals in accordance with an embodiment;

FIGS. 3(e) and 3(f) are flow charts of illustrative steps involved in performing an inverse continuous wavelet transform in accordance with some embodiments;

FIG. 4 is a block diagram of an illustrative continuous wavelet processing system in accordance with some embodiments;

FIGS. 5-7 show illustrative PPG signals and associated scalograms derived from signals obtained from various probe locations in accordance with some embodiments;

FIG. 8 shows an illustrative plot of respiration rate derived from a signal obtained from a more suitable probe location in accordance with some embodiments;

FIG. 9 shows an illustrative process for identifying the most suitable probe location for determining respiratory parameters in accordance with some embodiments;

FIGS. 10(a), 10(b), 10(c), and 10(d) show simplified block diagrams of flexible probes in accordance with some embodiments; and

FIGS. 11-15 show illustrative scalograms derived from signals obtained from standard and flexible probes positioned at various probe locations in accordance with some embodiments.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=I₀(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=R×(t) (8)

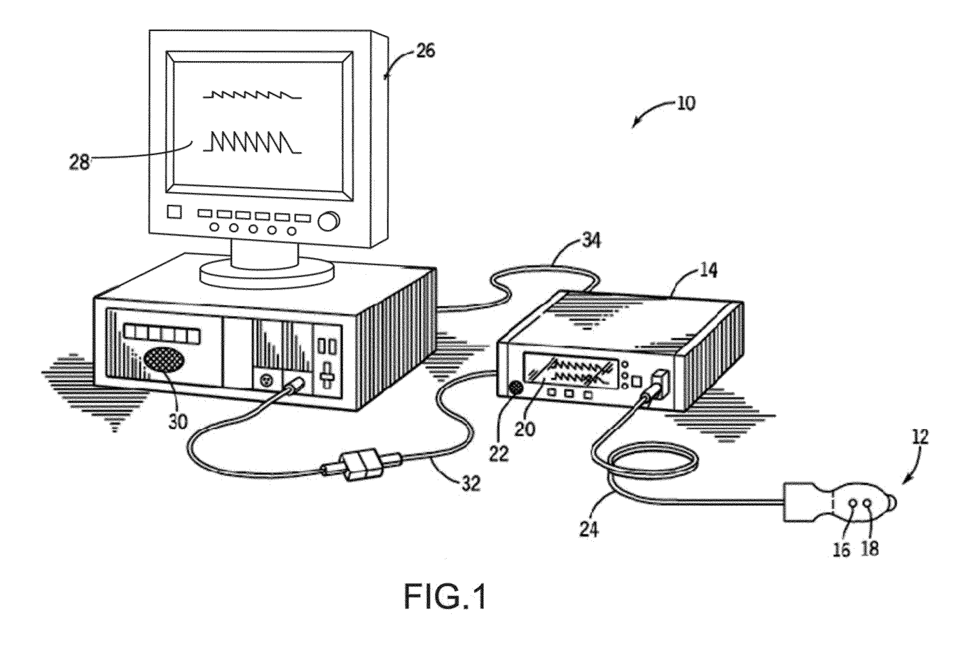

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

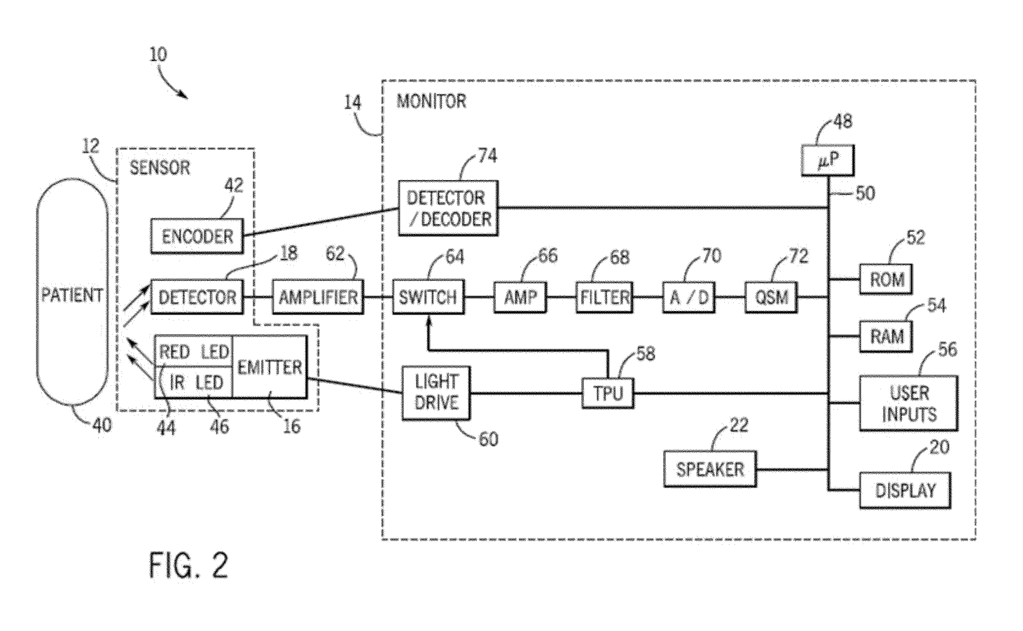

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be rescaled for useful purposes. One common rescaling is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of rescaling including, but not limited to, the original unscaled wavelet representation, linear rescaling, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}⁾^{²}^{/2})e^{t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

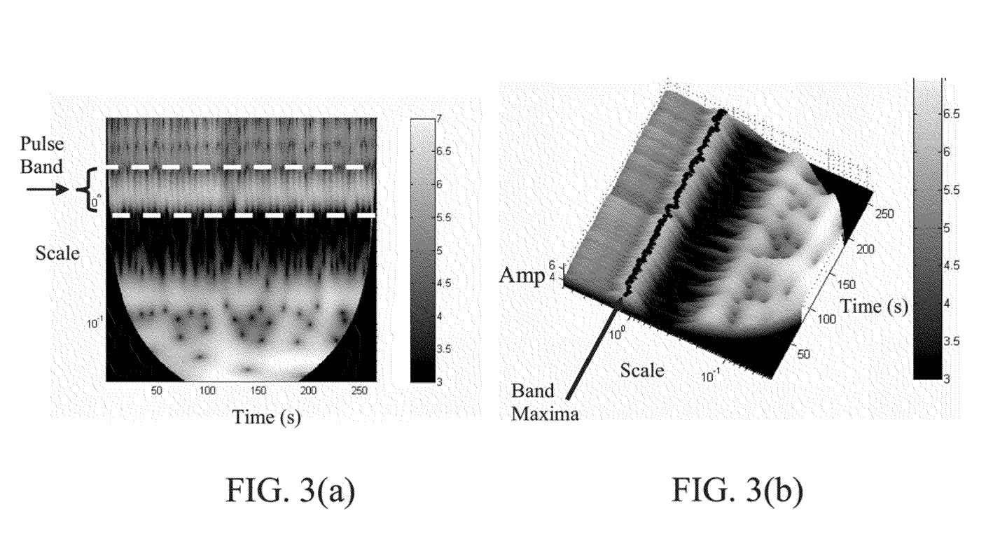

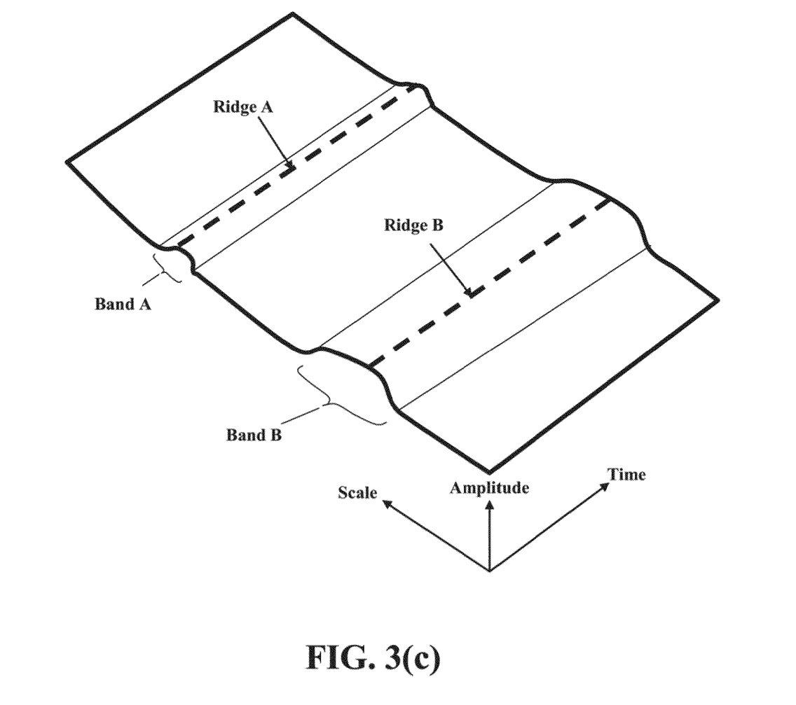

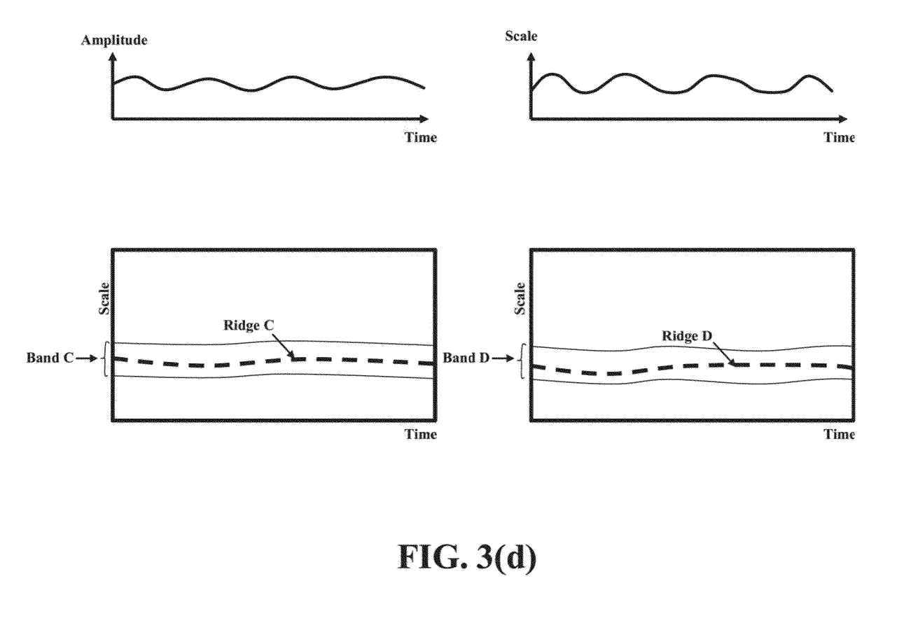

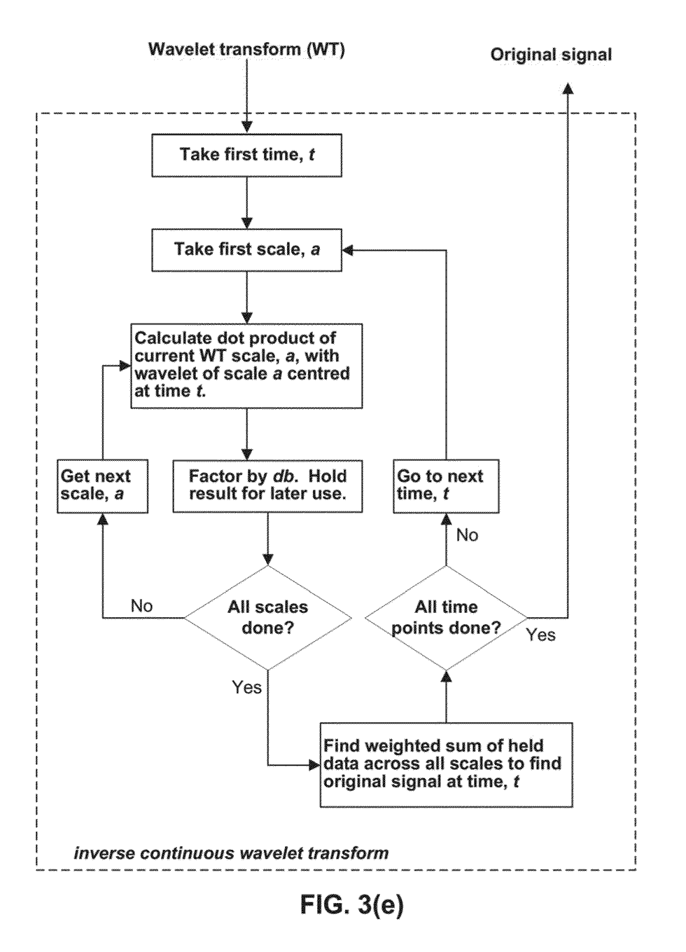

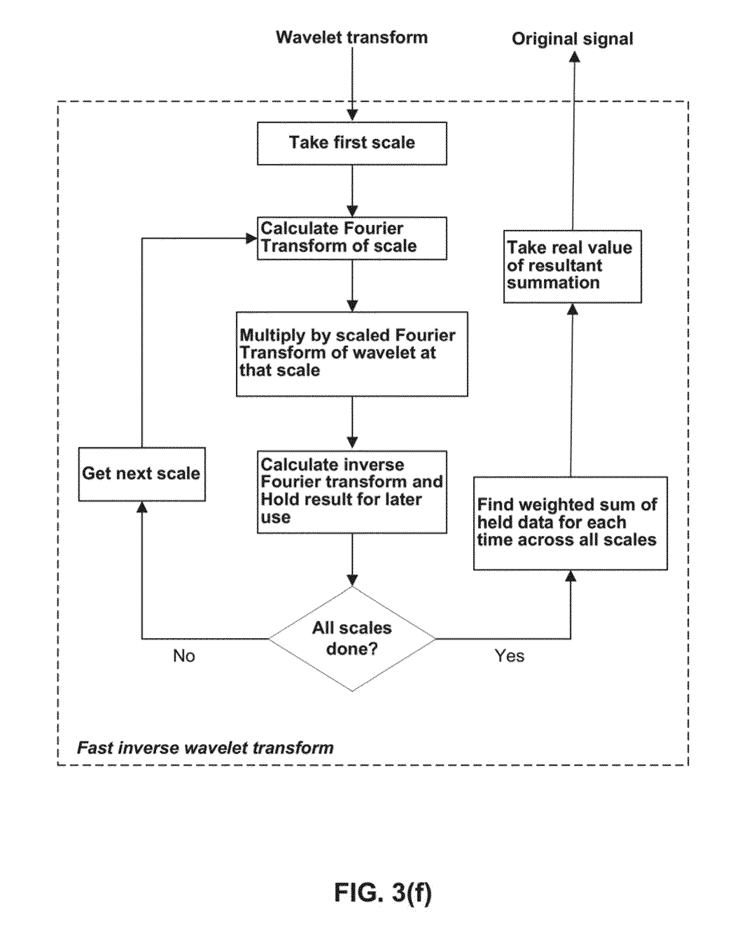

Pertinent repeating features in a signal give rise to a time-scale band in wavelet space or a rescaled wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable rescaling of the scalogram, such as that given in equation (11), the ridges found in wavelet space may be related to the instantaneous frequency of the signal. In this way, the pulse rate may be obtained from the PPG signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a rescaled wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

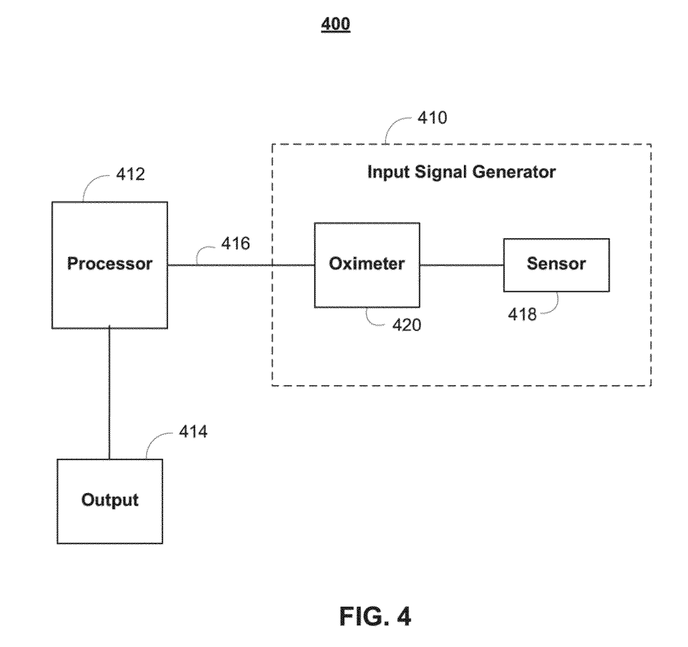

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

As described above, there may be several suitable probe locations for emitter 16 and detector 18 (FIG. 1). For example, a probe may be positioned on the finger, ear, toe, or forehead to determine an accurate value for SpO₂. Placing the probe at other locations on the patient's body may produce erroneous or inaccurate SpO₂ measurements (or the system may fail to report a value at all). Low perfused areas and highly pulsatile areas may both adversely affect a pulse oximeter's ability to compute an accurate SpO₂ value. Thus, these probe location are often avoided when trying to determine SpO₂.

When determining respiratory parameters, such as respiration rate and respiratory effort, some probe locations are more suitable than others. Probe locations where the modulation of the venous component dominates the PPG signal (or exceeds the arterial pulsatile component) may be used in an embodiment in order to enhance the detected PPG signal for determining respiratory parameters. Additionally or alternatively, locations that exhibit movement or motion associated with respiration may also be selected as more suitable probe locations to determine a patient's respiratory parameters. These locations may include, for example, a patient's collarbone, abdomen, side, chest (e.g., on or near the upper pectoral muscle), back, shoulder, or neck.

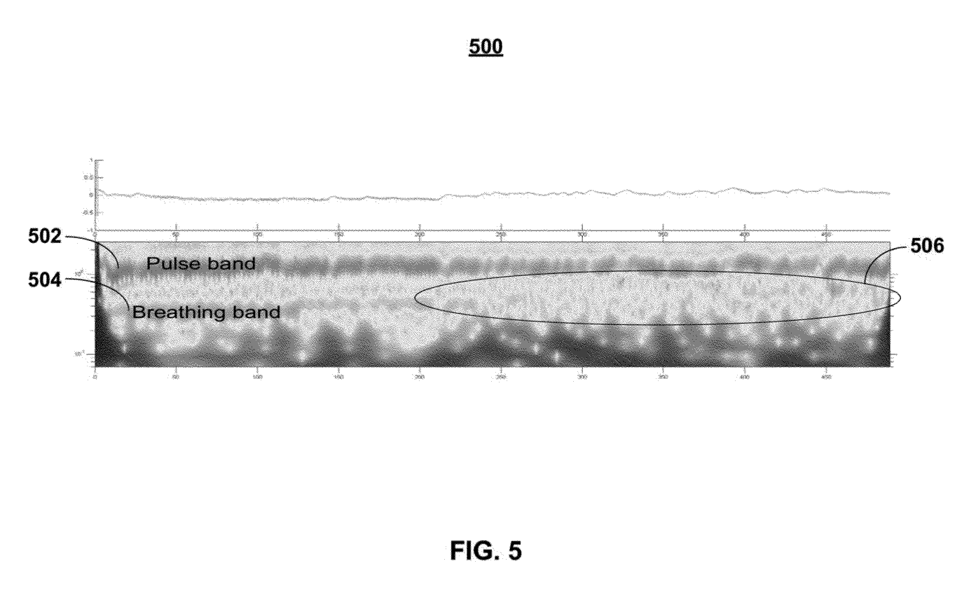

FIG. 5 shows illustrative PPG signal and associated scalogram signal 500. Signals 500 are derived from a probe placed on a finger of a patient. In the example shown in FIG. 5, the patient breathed at five different rates over a period of 480 seconds. First, the patient breathed at 20 breathes per minute (20 bpm) for 120 seconds, then the patient breathed at 25 bpm for 120 seconds, then the patient breathed at 30 bpm for 90 seconds, then the patient breathed at 35 bpm for 90 seconds, then the patient breathed at 40 bpm for 60 seconds. Thereafter, the patient breathed freely.

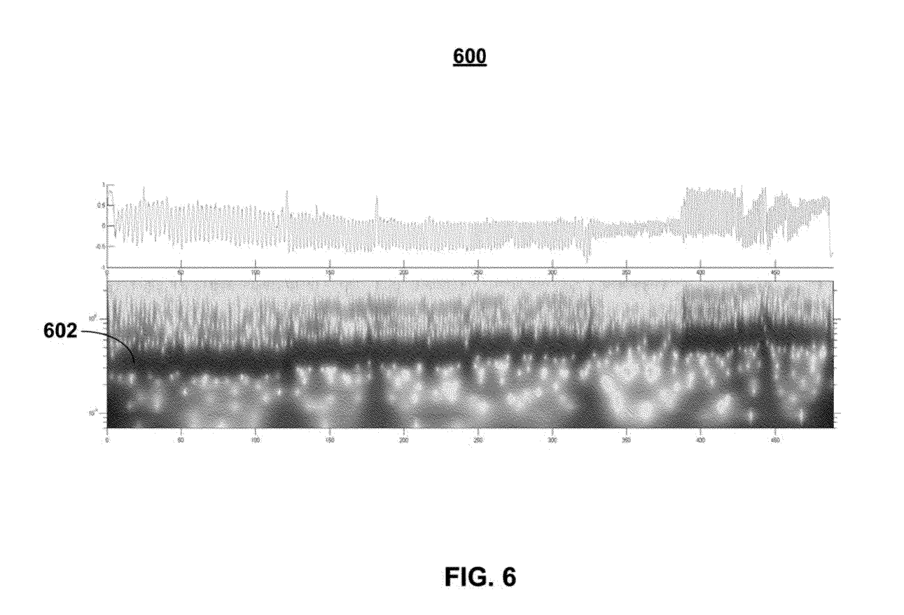

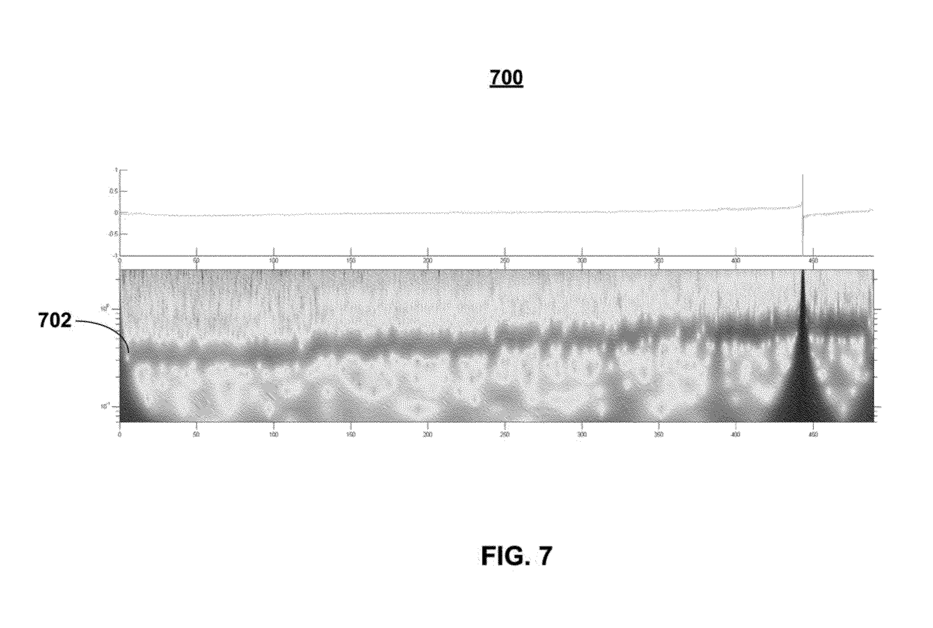

FIG. 6 shows illustrative PPG signal and associated scalogram signal 600. Signals 600 are derived from a probe placed on the collarbone of a patient collected simultaneously with the signals shown in FIG. 5. FIG. 7 shows illustrative PPG signal and associated scalogram signal 700. Signals 700 are derived from a probe placed on the chest (e.g., upper pectoral muscle) of a patient collected simultaneously with the signals shown in FIGS. 5 and 6.

Pulse band 502 and breathing band 504 can be seen in signals 500. As shown in signals 500, breathing band 504 becomes less distinct at higher respiration rates. This is shown by area 506 in the scalogram associated with the PPG signal in signals 500. In FIG. 6, which was collected using a probe placed on the patient's collarbone, the pulse band is not easily discernable. Breathing band 602, however, appears very distinct throughout the entire time period (i.e., even at high respiration rates). Similarly, in FIG. 7, which was collected using a probe placed on the patient's chest, the pulse band is not easily discernable. Breathing band 702, however, appears very distinct throughout the entire time period (i.e., even at high respiration rates). Similar scalograms with very distinct breathing bands may be obtained in other embodiments by positioning the probe at other locations, such as the lower chest, abdomen, side, shoulder, and back.

The scalograms shown in FIGS. 6 and 7 have distinct breathing bands because, at least in part, of the more suitable probe locations used. As previously described, traditional locations for pulse oximetry probes are selected so as to maximize the energy of the pulse band. Thus, probe locations yielding a strong arterial pulsatile component (e.g., the ear or finger) are traditionally used. To measure respiratory parameters, however, these traditional probe locations are less than ideal. Therefore, in an embodiment, probe locations may be selected where the modulation of the venous component dominates the PPG signal (or exceeds the arterial pulsatile component). Additionally or alternatively, locations that exhibit movement or motion associated with respiration may also be selected as more suitable probe locations to determine a patient's respiratory parameters at least in part because movement or motion in phase with a patient's respiration rate may enhance the breathing band (e.g., increase the energy associated with the breathing band) exhibited in the scalogram associated with the detected PPG signal.

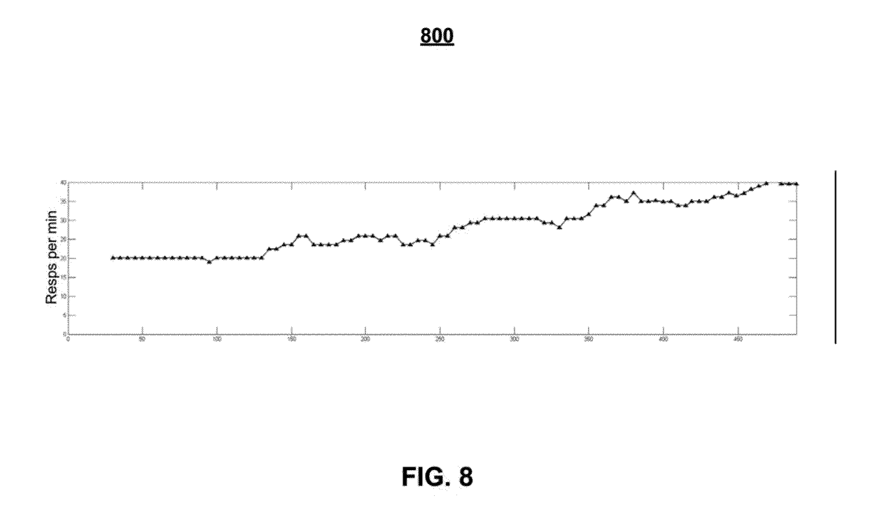

FIG. 8 shows plot 800 of a patient's respiration rate determined, at least in part, from breathing band 702 (FIG. 7) of signals 700 (FIG. 7). As explained in more detail in U.S. Patent App. Pub. No. 2006/0258921, which is hereby incorporated by reference herein in its entirety, the act of breathing may cause a breathing band to become present in a scalogram derived from a PPG signal. This breathing band may occur at or about the scale having a characteristic frequency that corresponds to the breathing frequency. Furthermore, the features within this band (e.g., the energy, amplitude, phase, or modulation) or the features within other bands of the scalogram may result from changes in breathing rate (or breathing effort) and therefore may be correlated with the patient's respiratory parameters and may be used to output the respiration rate of a patient.

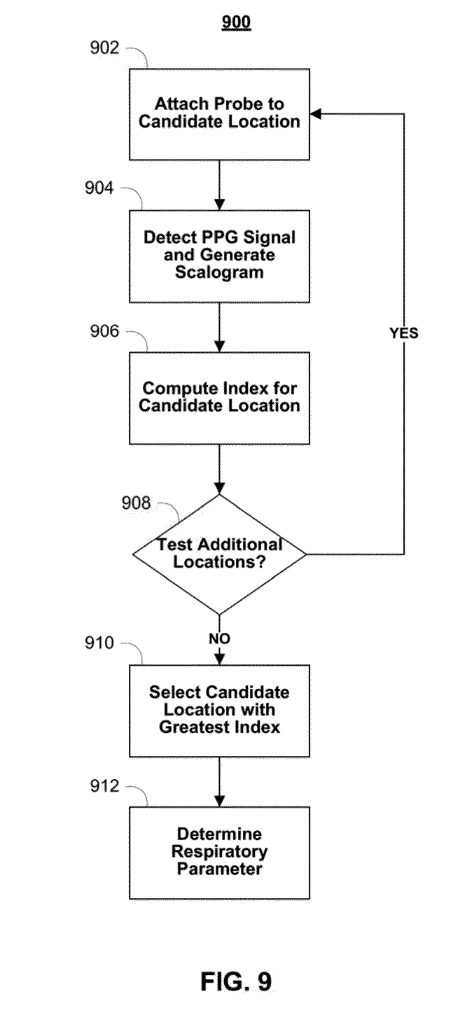

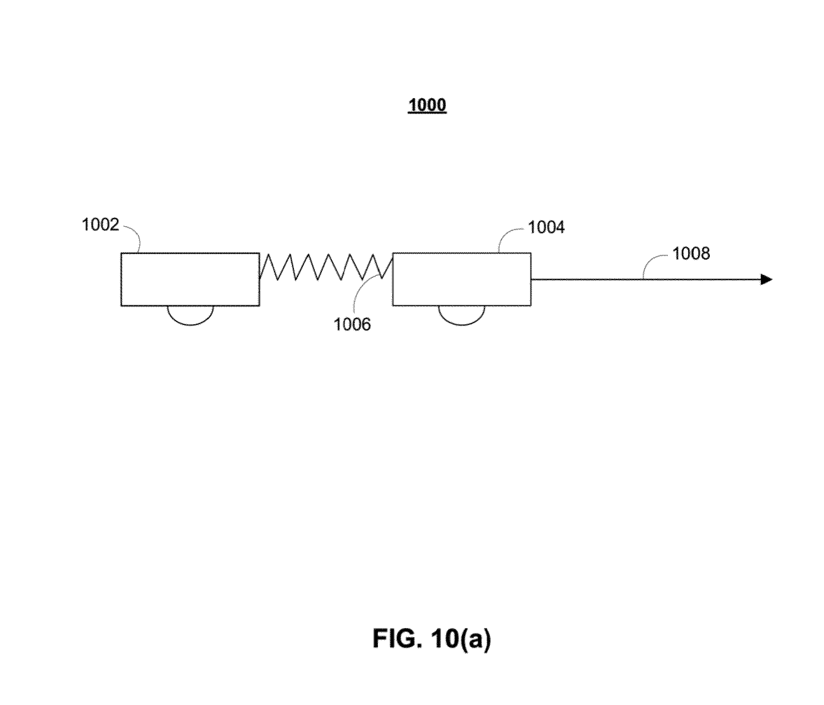

FIG. 9 shows illustrative process 900 for identifying the most suitable probe location for determining respiratory parameters, such as respiration rate and respiratory effort. At step 902, one or more probes (e.g., pulse oximetry probes) may be attached to candidate locations on the patient's body. For example, one or more instances of sensor 12 (FIG. 2) may be positioned on the collarbone, abdomen, side, chest (e.g., on or near the upper pectoral muscle), back, shoulder, or neck of patient 40 (FIG. 2). At step 904, a PPG signal may be detected from each probe and a scalogram corresponding to the detected PPG signal may be generated. For example, detector 18 (FIG. 2) of sensor 12 (FIG. 2) may detect energy (e.g., light) after passing through the tissue of patient 40. At step 906, an index may be computed for the current candidate location or locations. The index may also be outputted to a user (e.g., a physician or technician) in visual or audible form. In some embodiments, the computed index may be proportional to, for example, the breathing band energy of the generated scalogram, the ratio of the breathing band energy to the pulse band energy of the generated scalogram, or inversely proportional to the pulse band energy of the generated scalogram. The index may also be proportional to some characteristic of the detected PPG signal itself (e.g., before or instead of performing a wavelet decomposition).

In general, the computed index may be related to or reflect the suitability of that particular probe location for determining at least one respiratory characteristic or parameter, such as respiration rate or respiratory effort. A high index may indicate that that particular location may have low pulse band energy (because a strong pulse band may distort the breathing band or make it more difficult to accurately detect), high breathing band energy, or both low pulse band energy and high breathing band energy. A high index may additionally or alternatively indicate that a consistent energy level in a band has been maintained over a period of time. Because different locations may be more or less suitable than other locations for each patient, multiple locations may be tested and the most suitable location selected as the best probe location. At step 908, a determination is made whether additional locations are to be tested. If there are additional locations to test, illustrative process 900 returns to step 902 to test the next candidate location.

At step 910, the candidate location with the greatest index may be chosen as the best probe location for determining respiratory characteristics or parameters. At step 912, at least one respiratory characteristic or parameter may be determined by positioning a probe at the selected location associated with the greatest index. For example, one or more of respiration rate or respiratory effort may be determined by generating a scalogram from the PPG signal detected at step 904. As described above, for example, the breathing band may be isolated in the scalogram and used to determine various respiratory parameters. Other techniques (other than an analysis of the breathing band in the scalogram) may also be used to determine respiratory parameters in some embodiments. The more suitable probe locations described in the present disclosure may also be used to accurately determine respiratory parameters using, for example, frequency modulation techniques, amplitude modulation techniques, correlation techniques (e.g., correlation with non-respiratory signals), cross-spectral analyses, baseline analyses, or any combination of the foregoing. Any suitable filtering techniques (e.g., low-pass filtering, Kalman filtering, or least mean square (LMS) filtering) may also be used to determine respiratory parameters from a detected PPG signal using the more suitable probe locations described in the present disclosure. For example, a low-pass filter may first remove pulse components from the detected signal leaving breathing components behind. The breathing components of the signal may then be analyzed (e.g., by analyzing baseline changes), from which respiratory parameters may be determined.

In some embodiments, a fixed positive number N locations are tested during process 900. The location with the greatest index is then used as the most suitable probe location to determine respiratory parameters. In other embodiments, there is no predetermined number of locations tested. For example, in an embodiment, new locations may be tested until a location with a desired index (e.g., above a predetermined or dynamic threshold) is discovered. For example, the index of each tested location may be compared to a user-defined or system-defined threshold suitability index. If a particular location meets or exceeds the threshold suitability index, then illustrative process 900 may continue to step 912 to determine a respiratory parameter at that location in some embodiments. An indication (e.g., an audible or visual indication) may also be provided when a suitable location is discovered.

In an embodiment, all or a part of illustrative process 900 may be automated. For example, in an embodiment, a plurality of wired or wireless probes (as described in more detail below) may be automatically attached to a patient at a plurality of candidate locations corresponding to potential suitable locations for determining respiratory parameters. The probes may be attached manually by a physician or technician or automatically using a robotic arm, mechanical scanner, or the like. If automatic or mechanical positioning of probes is desired, an image of the patient's body may be first taken and used to determine suitable coordinate locations for probe placement. In some embodiments, candidate locations are tested serially one after another until a suitable location is discovered (or all candidate locations have been tested). In other embodiments, more than one candidate location is tested simultaneously.

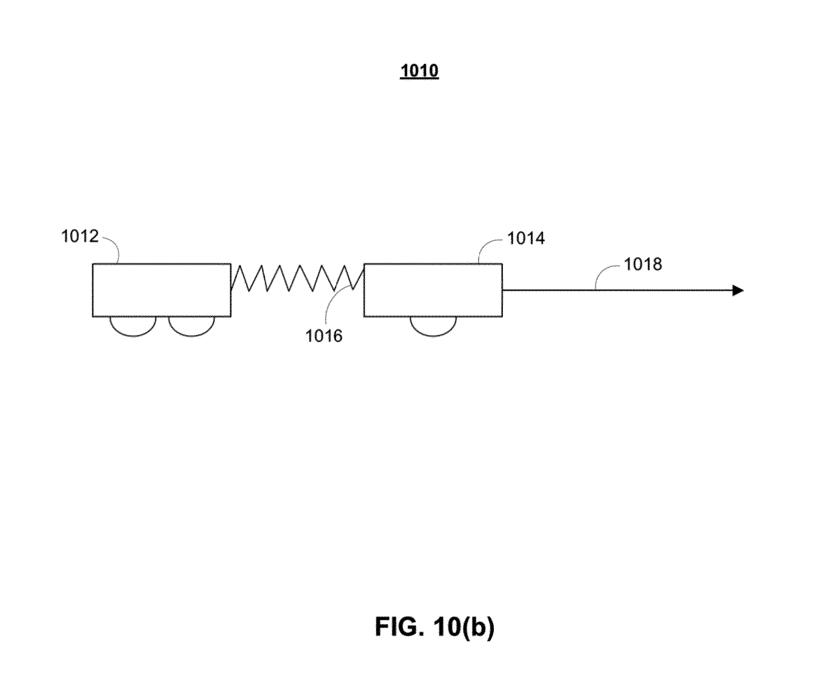

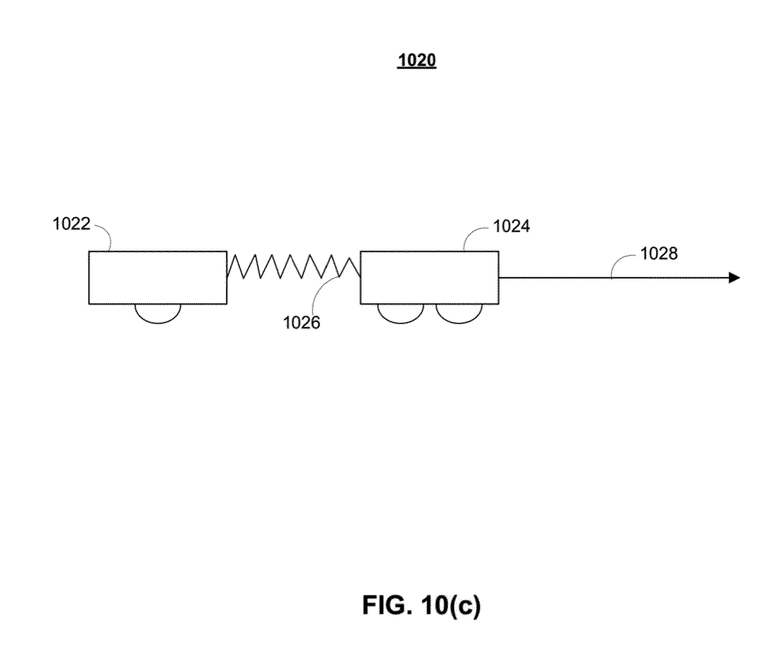

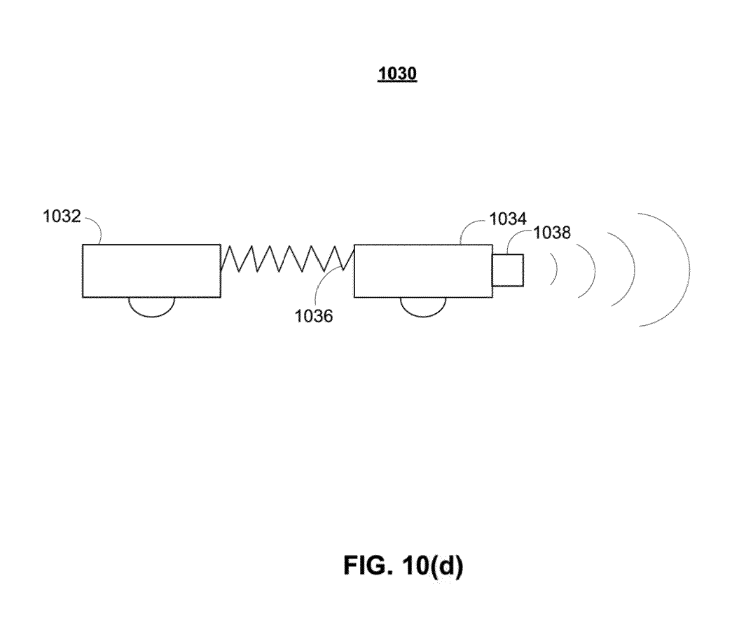

FIGS. 10(a), 10(b), and 10(c) show three enhanced probes for use in determining a patient's respiratory parameters. In general, these enhanced probes (sometimes referred to as “flexible probes” herein) allow for the natural movement due to respiration at certain sites on the patient's body. A patient's natural movement due to respiration may enhance the signal detected by the probe for use in determining respiratory parameters, such as respiration rate and respiratory effort. For example, movement in phase with a patient's respiration may enhance the respiration components of a detected PPG signal.

FIG. 10(a) shows flexible probe 1000. Probe 1000 includes energy emitting source 1002 (e.g., a light emitting source such as an LED) separated from energy detector or sensor 1004 (e.g., a photodetector) by flexible member 1006. Connecting energy emitting source 1002 to energy detector or sensor 1004 by flexible member 1006 allows for natural movement between energy emitting source 1002 and energy detector or sensor 1004. As an example, probe 1000 may be positioned on the patient's chest (e.g., upper pectoral muscle). As the patient breathes, movement of the patient's chest may be detected between energy emitting source 1002 and energy detector or sensor 1004. This movement, which may be substantially in phase with the patient's respiration, may enhance the breathing components of the signal detected by energy detector or sensor 1004.

Flexible member 1006 may be composed of any suitably flexible material, including, for example, an elastoplastic, rubber, synthetic polymer, coil, spring, wire, or any combination of the foregoing. Regardless of the type of material used, flexible member 1006 may permit natural movement between energy emitting source 1002 and energy detector or sensor 1004. Lead 1008 may send the signal detected by energy detector or sensor 1004 to a parent device (not shown). For example, lead 1008 may be connected to a pulse oximetry system or other physiological characteristic monitoring system.

Although the example shown in FIG. 10(a) shows only one energy emitting source separated from one energy detector or sensor by a single flexible member, in other embodiments, more than one energy emitting source is separated from one or more energy detector or sensor by one or more flexible member. Any number of energy emitting sources, energy detectors or sensors, and/or flexible members may be used in other embodiments. For example, FIG. 10(b) shows flexible probe 1010. Probe 1010 includes two energy emitting sources 1012 connected to energy detector or sensor 1014 by flexible member 1016. In an embodiment, energy emitting sources 1012 may include, for example, light emitting sources at red and infrared wavelength. Lead 1018 may send the signal detected by energy detector or sensor 1004 to a parent device (not shown). For example, lead 1018 may be connected to a pulse oximetry system or other physiological characteristic monitoring system.

In an embodiment, at least one energy emitting source may be rigidly coupled to an energy detector or sensor while at least one other energy emitting source may be separated by the detector or sensor by a flexible member. As shown in FIG. 10(c), probe 1020 includes energy emitting sources 1022 connected to housing 1024 by flexible member 1026. Housing 1024 may be a rigid housing that includes at least one energy emitting source and at least one energy detector or sensor in the same housing. In this way, at least one energy emitting source (e.g., energy emitting source 1022) may be separated from the energy detector or sensor by flexible member 1026 while another energy emitting source may be rigidly coupled to the energy detector or sensor in housing 1024. The energy emitting source flexibly coupled to the energy detector or sensor may include a red (or infrared) light emitting source, while the energy emitting source rigidly coupled to the energy detector or sensor may include an infrared (or red) light emitting source. This may allow the movement portion of the signal detected by the energy detector or sensor to be differentiated from other components of the detected signal (e.g., pulse components, such as cyclical venous inflow or outflow). Lead 1028 may send the signal detected by energy detector or sensor to a parent device (not shown). For example, lead 1028 may be coupled to a pulse oximetry system or other physiological characteristic monitoring system.

In an embodiment, the flexible probe of the present disclosure may include multiple energy detectors or sensors (e.g., photodetectors) arranged in a flexible array that covers a local area over a patient's body within the vicinity of one or more energy emitting source. In this way, a number of signals indicative of motion may be detected from a local area. As described above, at least one of the energy detectors or sensors may be rigidly coupled (e.g., placed on the same rigid substrate or included in the same rigid housing) as the energy emitting source. The detector or sensor rigidly coupled to the energy emitting source may be configured to determine SpO₂ while the remaining energy detectors or sensors in the flexible array may be configured to determine one or more respiratory parameters.

In an embodiment, the standard or flexible probes of the present disclosure may be wirelessly coupled to a parent device (e.g., a pulse oximetry system or other physiological characteristic monitoring system). The at least one wireless probe may be attached (e.g., using removable adhesive, gel, or a suction cup attachment) to a patient at a suitable location for determining respiratory parameters. In this way, no extra lead may be required to monitor respiratory parameters. As shown in FIG. 10(d), wireless probe 1030 includes at least one energy emitting source 1032 separated from at least one energy detector or sensor 1034 by flexible member 1036. Wireless transmission device 1038 (e.g., a wireless transceiver or wireless network interface) may replace the lead connecting wireless probe 1030 to its parent device. Wireless probe 1030 may then wirelessly transmit and receive data and instructions to and from the parent device.

Multiple wireless probes may also be used in some embodiments. One of more of the wireless probes may be pulse oximeter probes. One wireless probe may be positioned at a more traditional location for pulse oximetry (e.g., on a finger) and used to determine a patient's blood oxygen saturation (referred to as a “SpO₂” measurement), while another wireless probe may be placed at a more suitable location for determining respiratory parameters. Multiple additional wireless probes may also be positioned at various other locations to determine various other physiological parameters. For example, one wireless probe may be positioned on the finger and used to determine SpO₂, one wireless probe may be positioned on the abdomen and used to determine respiration rate, one wireless probe may be positioned on the chest and used to determine respiratory effort, and one wireless probe may be positioned on the ear (or finger) and used to determine blood pressure.

The flexible members of any of the probes described above may permit movement in all directions or may permit movement in only certain directions or certain planes of motion. For example, one or more of the probes described above may include a flexible member that is restrained from moving in one or more planes of motion. A pivot or hinge may be incorporated into the flexible member and used to restrain motion in the one or more planes of motion. For example, the pivot or hinge may be used to restrain horizontal motion (e.g., between the energy emitting source and energy detector or sensor) and allow for vertical motion (or restrain vertical motion and allow for horizontal motion). The planes of permitted and restrained motion may be used increase the resolution or energy associated with the respiratory components of the detected signal. Multiple planes of motion may be arranged in such a way (e.g., in orthogonal directions) so as to enable improved resolution or improved identification of the respiratory components of the detected signal.

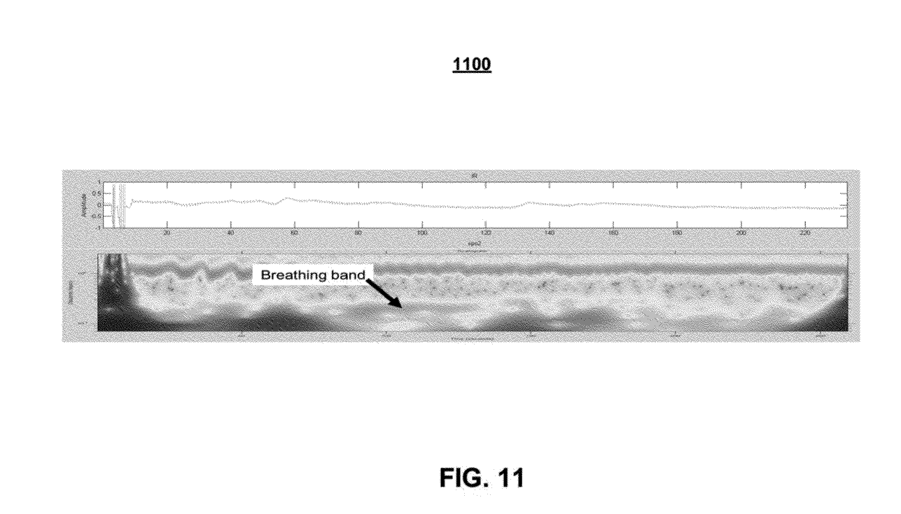

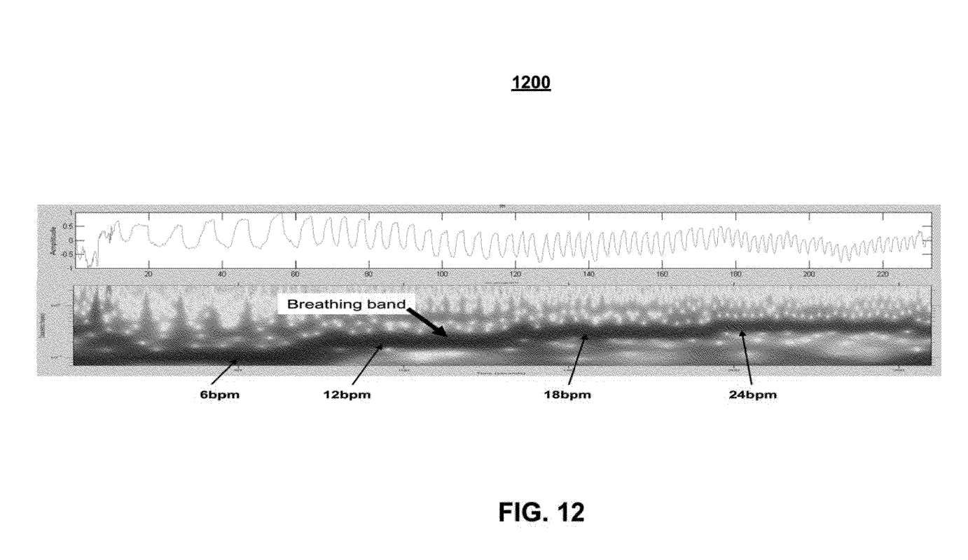

FIGS. 11-13 show illustrative scalograms derived from signals obtained from standard and flexible probes positioned at various probe locations in accordance with some embodiments. FIG. 11 shows PPG signal and scalogram signal 1100 taken at a finger site using a standard probe. FIG. 12 shows PPG signal and scalogram signal 1200 taken at a chest site using a flexible probe. The signals in FIGS. 11 and 12 were collected at the same time from the same patient. First, the patient breathed at 6 breathes per minute (20 bpm) for 60 seconds, then the patient breathed at 12 bpm for 60 seconds, then the patient breathed at 18 bpm for 60 seconds, then the patient breathed at 24 bpm for 60 seconds. Thereafter, the patient breathed freely.

From a comparison of the scalograms shown in FIGS. 11 and 12, the flexible probe positioned on the chest yielded a signal enhancement over the standard finger probe. More specifically, the breathing components of the signal are stronger in the flexible probe positioned on the patient's chest than in the standard probe positioned on the patient's finger. As described above, this is due, at least in part, to the natural movement associated with the patient's respiration. The patient's natural movement, which is substantially in phase with the patient's respiration, acts to enhance the breathing components of the detected signal. The use of the flexible probes shown in FIGS. 10(a), 10(b), 10(c), and 10(d) allows for this natural movement to manifest itself in the detected signal, resulting in an improved signal for the determination of respiratory parameters.

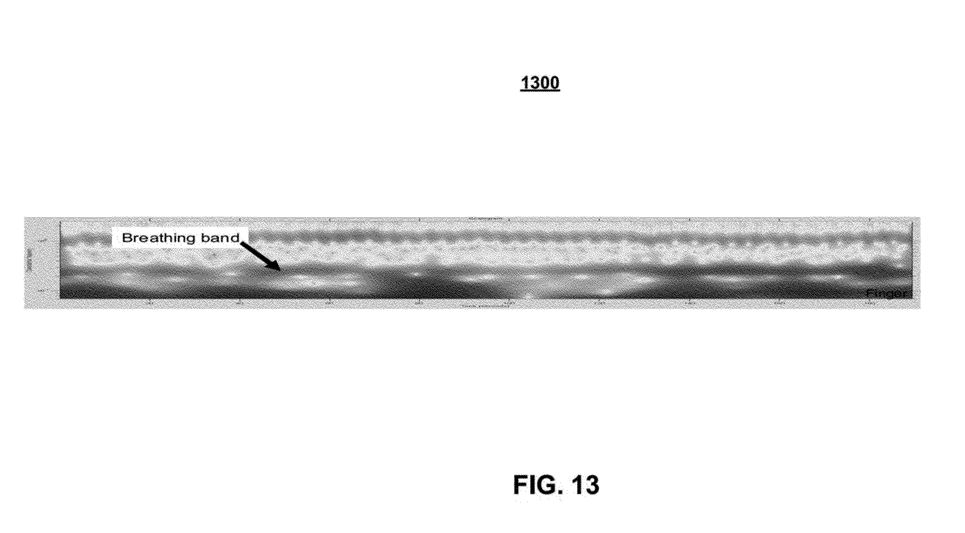

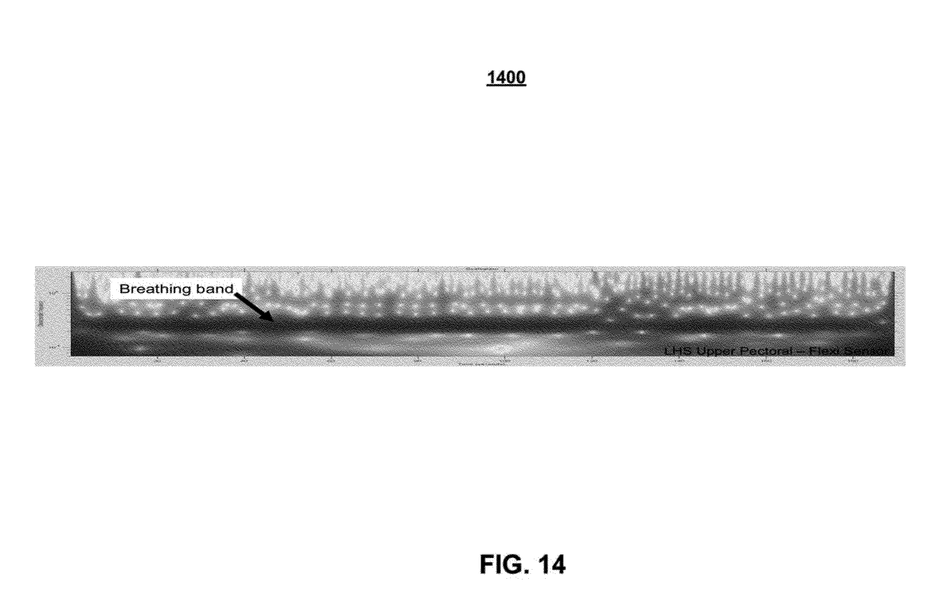

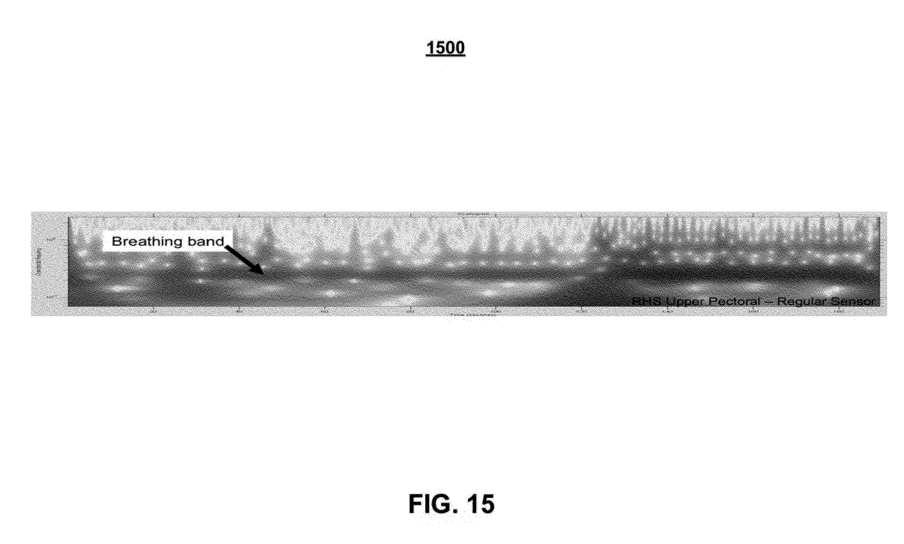

The flexible probe of the present disclosure may provide enhanced breathing band signals even at varying levels of respiratory effort. FIGS. 13, 14, and 15 show signals derived from a patient breathing at a constant rate. At 120 seconds, the patient began breathing against a resistance, which increased the patient's respiratory effort. The increase in effort is only slightly noticeable in scalogram 1300 of FIG. 13, which was taken from a standard finger probe. As shown in scalogram 1400 of FIG. 14, however, the flexible probe placed on the chest yields a signal with a strong breathing band before and after the increase in the patient's respiratory effort. As such, the flexible probe positioned on the chest did not differentiate the increase in respiratory effort very well. Thus, in some embodiments, the standard probe may offer a more suitable signal for determining respiratory effort, while the flexible probe may offer a more suitable signal for determining respiration rate. This can be clearly seen in scalogram 1500 of FIG. 15, which was derived from a standard probe positioned on the patient's chest. A distinct change in the breathing band energy can be seen starting at 120 seconds in scalogram 1500.

Although the flexible probe of the present disclosure is often described herein as being positioned on the upper pectoral muscle of the chest, in some embodiments the flexible probe may be positioned on the chest wall, the shoulder, the collarbone, the side of chest, around the diaphragm, or any other location where natural movement due to respiration is exhibited or may be detected. If attached to the chest or abdomen, a chest band or abdomen band may be used to secure the probe to the patient. The probes in this case may be wireless probes. The housing of the probe (including the circuitry and electronics associated with the probe) may be at least partially housed in the chest or abdomen band. Similar bands may be used on other parts of the body as well.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems, and apparatuses, but is intended to include variations to and modifications thereof which are within the spirit of the following claims.

What is claimed is:

1. A system for determining a respiratory parameter of a patient, comprising:
one or more probes configured to receive:
a first photoplethysmograph (PPG) signal from a first probe location, wherein the first PPG signal comprises a first respiratory component; and
a second PPG signal from a second probe location, wherein the second PPG signal comprises a second respiratory component; and
a processor configured to:
compute a first index for the first probe location, wherein the first index is based at least in part on the first respiratory component, and wherein the first index is indicative of energy level consistency of the first respiratory component over time;
compute a second index for the second probe location, wherein the second index is based at least in part on the second respiratory component, and wherein the second index is indicative of energy level consistency of the second respiratory component over time; and
select, based at least in part on the first index and the second index, one of the first probe location and the second probe location for determining at least one respiratory parameter.

2. The system of claim 1 wherein the processor is additionally configured to determine the at least one respiratory parameter at the selected probe location.

3. The system of claim 1 wherein the at least one respiratory parameter is selected from the group consisting of respiration rate and respiratory effort.

4. The system of claim 1 wherein the first location is selected from the group consisting of a collarbone, abdomen, side, chest, back, shoulder, and neck.

5. The system of claim 1 wherein the processor is configured to compute a first index for the first probe location by:
performing a continuous wavelet transform on the first PPG signal to produce a first transformed signal; and
generating a scalogram based at least in part on the first transformed signal.

6. The system of claim 5 wherein the processor is further configured to:
identify a breathing band in the scalogram; and
determine the energy associated with the breathing band.

7. The system of claim 6 wherein the first index is proportionally related to the determined energy associated with the breathing band.

8. The system of claim 6 wherein the processor is further configured to:
identify a pulse band in the scalogram; and
determine the energy associated with the pulse band.

9. The system of claim 8 wherein the first index is proportionally related to the ratio of the determined energy associated with the breathing band to the determined energy associated with the pulse band.

10. The system of claim 1 wherein the first probe location and the second probe location exhibit natural movement due to respiration of the patient.

11. A method for determining a respiratory parameter of a patient, comprising:
receiving a first photoplethysmograph (PPG) signal from a probe attached to a subject at a first probe location, wherein the first PPG signal comprises a first respiratory component;
computing a first index for the first probe location, wherein the first index is based at least in part on the first respiratory component, and wherein the first index is indicative of energy level consistency of the first respiratory component over time;
receiving a second PPG signal from a probe attached to a subject at a second probe location, wherein the second probe location is different than the first probe location, and wherein the second PPG signal comprises a second respiratory component;
computing a second index for the second probe location, wherein the second index is based at least in part on the second respiratory component, and wherein the second index is indicative of energy level consistency of the second respiratory component over time; and
selecting, based at least in part on the first index and the second index, one of the first probe location and the second probe location for determining at least one respiratory parameter.

12. The method of claim 11 further comprising determining the at least one respiratory parameter at the selected probe location.

13. The method of claim 11 wherein the at least one respiratory parameter is selected from the group consisting of respiration rate and respiratory effort.

14. The method of claim 11 wherein the first location is selected from the group consisting of a collarbone, abdomen, side, chest, back, shoulder, and neck.

15. The method of claim 11 wherein computing a first index for the first probe location comprises:
performing a continuous wavelet transform on the first PPG signal to produce a first transformed signal; and
generating a scalogram based at least in part on the first transformed signal.

16. The method of claim 15 further comprising:
identifying a breathing band in the scalogram; and
determining the energy associated with the breathing band.

17. The method of claim 16 wherein the first index is proportionally related to the determined energy associated with the breathing band.

18. The method of claim 16 further comprising:
identifying a pulse band in the scalogram; and
determining the energy associated with the pulse band.

19. The method of claim 18 wherein the first index is proportionally related to the ratio of the determined energy associated with the breathing band to the determined energy associated with the pulse band.

20. The method of claim 11 wherein the first probe location and the second probe location exhibit natural movement due to respiration of the patient.
